(19) United States
(12) Patent Application Publication
(10) Pub. No.: US 2003/0190379 A1
(43) Pub. Date: Oct. 9, 2003

(54) Dressing for treating burns

(76) Inventor: Du Ben Hai, Beijing (CN)

Correspondence Address: Robert Samuel Smith, 1263 EMORY ST., SAN JOSE, CA, 95126, US

(21) Appl. No.: 10/115,804

(22) Filed: Apr. 3, 2002

(51) Int. Cl. A61K 35/78; A61K 9/70

(52) U.S. Cl. 424/741; 424/773; 424/775

(57) ABSTRACT

A salve for application to a burn wound to support healing the wound including a mixture of healing agents which consists of Radix Scutellariae, Cortex Phellodendri, Rhizoma Coptidis mixed according to the ratio by weight of 1:1:1 The mixture is ground to pass through a 180 mesh screen. A carrier is formulated by dissolving beeswax in sesame oil according to the ratio of 10 grams of beeswax in 100 milliliters of sesame oil. The ground mixture is added to the sesame oil containing bee's wax according to the ratio of 10.7 grams of mixture to 100 milliliters of oil so as to form a salve. A preferred method of treatment is to spread the salve on a gauze pad and then to lay the pad over the wound.

### FIELD OF THE INVENTION

This invention relates to properties of dressings for treating burns regarding the method of preparation and particularly to a preparation whose method of preparation provides superior results in terms of accelerating the healing process and reducing the discomfort of the patient.

### BACKGROUND AND INFORMATION DISCLOSURE

The healing process of wounds that result from burns involves migration of metabolic products (excreta) from the wound and migration of healing agents (drugs) into the wound.

The metabolic products include dead cells that have been destroyed by the initial assault (burn), antibodies (e.g., white corpuscles of the blood) that the body transports to the site of the wound to fight infection, and the vehicle (fluids) that the body pours into the burn region for the purpose of “washing out” the dead cells and antibodies.

Healing agents in drugs that migrate into the wound to promote healing include bactericides, fungicides, analgesics (to deaden pain), agents that promote the growth of collagen. Medications that have been developed to treat bums generally contain the healing agents and a vehicle (ointment() that retains the healing agents at the burn site. Various fluids and semi fluids have been used for this purpose. Such fluids include sesame oil and semi fluids include vaseline.

U.S. Pat. No. 5,405,608 to Rongxiang Xu discloses a system of “moist therapy” in which the medicament is a viscous oil extract. The extract is prepared by adding Huangquin, Huanglian, Huangbai, earthworm, poppy seed, and bees wax to sesame oil heated to 120° C. stirring for about 45 minutes, and then filtering the liquid. The filtrate (medication) contains about 85-97% sesame oil.

U.S. Pat. No. 5,817,322 to Rongxiang Xu discloses a netted framework structure of beeswax saturated with sesame oil containing healing agents. The concoction is applied to the wound where it forms a “netted framework” through which the healing agents migrate to the wound.

In spite of substantial progress in the treatment of bum wounds, there continues to be demand for faster relief from pain and healing of the burn wounds.

### SUMMARY OF THE INVENTION

It is an object of this invention to provide an improved method for treating burn wounds in terms of accelerating the rate of healing and reducing the degree of discomfort suffered by the patient.

It is another object of this invention to reduce the amount of attention required to care for the wound in terms of a reduced number of times taken to replace a dressing and in terms of lengthening the periods between changing the dressing.

This invention is directed toward a salve in which the healing agents in the salve are characterized by a particle size selected to maximize the benefits of the healing agent. The method of manufacturing the salve involves the step of reducing the particle size of the healing agents by methods such as grinding until the product will pass through a 180-mesh sieve. A carrier is then added to the grinding in a ratio of carrier to grindings in a ratio that reduces the consistency of the product to a paste.

The preferred method of application is to spread the salve over a gauze bandage. The bandage is gently laid onto the surface of the wound.

With either method of application, it is observed that the efficacy of the dressing prepared according to the invention is measurably improved compared to the methods of the prior art.

For example, in terms of comparative ability of dressings to alleviate pain, dressing applied according to the “moist process” reported in the prior art required changing the dressing every four to six hours whereas the dressing of the present invention retains its effectiveness for periods of three to four days without requiring a fresh change of the dressing.

Another advantage of the present invention is the absence of itching of the wound area during the healing process. In addition to improving the comfort of the patient, the absence of itchiing results in the patient refraining from scratching the wound. Such scratching of the wound results in scarring that is otherwise eliminated by the practice of this invention.

While I do not wish to be bound by theory, it is believed that the improved curative power of the invention relates to an improved control of release of the healing agent compared to the prior art. The practice of extraction, as disclosed in the “moist process” of the prior art, provides an “ointment-like base, in which the drug is carried across the interface between the carrier and wound by diffusion of the finely divided drug through the carrier. In the present invention, migration of the healing agent into the wound depends on direct contact between the particles of the healing agent and the wound. According to this model of the invention, the selection of a large particle size of the healing agent in direct contact with the wound provides immediate access of the wound to the healing agent.

### BRIEF DESCRIPTION OF THE DRAWING

FIG. 1 is a flow chart of the method of the invention.

### DESCRIPTION OF A BEST MODE

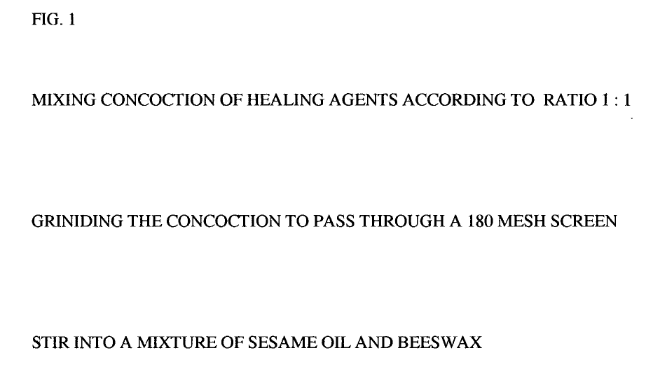

Turning now to a discussion of the drawings, FIG. 1 is a flow chart of the method of this invention for preparing a salve applied to the treatment of burns.

The composition includes:

Healing agent: (Composition A) (content by % weight)

(1) Radix Scutellariae—about 33

(2) Cortex Phellodendri—about 33

(3) Rhizoma Coptidis—about 33

Grind the mixture of agents to pass through a 180 mesh screen thereby providing composition A.

The carrier is Beeswax in sesame oil.

Heat sesame oil to 80°

Add 10.7 grams of beeswax per 100 ml of sesame oil then allow the oil-beeswax mix to cool to 25° C.

Add and stir 10.7 grams of Composition A per 100 milliliters of sesame/beeswax thereby producing a salve having the consistency of a salve.

While the salve may be applied directly to the wound with a spatula, a preferred method of application is to spread the salve onto a gauze pad to form a layer about 2 millimeters thick. The pad is then applied directly over the wound.

Experience in practicing the method of this invention has been noticeable reduction of swelling and pain of the wound within 4 to 5 seconds after application of the dressing.

Elimination of itching during the healing process of second degree burns is observed.

The dressing need only be changed every three days compared to practice of the prior art where it is required to change the dressing every four to six hours. Reduction of the required frequency to change the dressing greatly improves comfort and convenience to the patient during the healing process.

Variations and modifications of the invention may be contemplated which are within the scope of the invention. I therefore wish to define the scope of my invention by the appended claims.

What is claimed is:

1. A salve for application to a burn wound to support healing the wound which comprises:
a mixture of healing agents which consists of Radix Scutellariae, Cortex Phellodendri, Rhizoma Coptidis;
each said healing agent present in said mixture in the amount of about 33% by weight;
said mixture of agents ground to pass through a 180 mesh screen;
a carrier formulated by dissolving beeswax in sesame oil according to the ratio of 10 grams of beeswax in 100 milliliters of sesame oil;
said mixture stirred into said carrier according to the ratio of 10.7 grams of said mixture to 100 milliliters of sesame oil.

2. A method for preparing a salve for application to a burn wound which comprises:
heating sesame oil to a temperature of about 80° C.;
adding beeswax to said heated sesame oil in the amount of about 10 grams of beeswax per 100 milliliters of sesame oil after which, said sesame oil containing beeswax is permitted to cool to aboout 25° C.;
mixing together by weight one part Radix Scutellariae, one part Cortex Phellodendri, and one part Rhizoma Coptidis thereby forming a healing agent mixture;
grinding said healing mixture sufficiently to permit passing said mixture through a 180 mesh screen;
adding said healing agent mixture to said sesame oil containing beeswax according to the ratio about 10.7 grams of said mixture to 100 milliliters of sesame oil containing bees wax.

3. A method for treating a burn wound which comprises:
heating sesame oil to a temperature of about 80° C.;
mixing together by weight one part Radix Scutellariae, one part Cortex Phellodendri, and one part Rhizoma Coptidis thereby forming a healing agent mixture;
grinding said healing mixture sufficiently to permit passing said mixture through a 180 mesh screen;
adding said healing agent mixture to said sesame oil containing beeswax according to the ratio about 10.7 grams of said mixture to 100 milliliters of sesame oil containing bees wax,
spreading a layer of said salve onto a pad;
laying said pad with said layer onto said burn wound.

4. A dressing for application to a burn wound which comprises
the salve of claim 1;
a pad;
a layer of said salve spread on one side of said pad to a thickness of said layer of about two millimeters.
